(19) United States
(12) Patent Application Publication
(10) Pub. No.: US 2018/0344973 A1
(43) Pub. Date: Dec. 6, 2018

(54) CATHETER WITH DEDICATED FLUSHING PORT

(71) Applicant: Jane ALLEN, Cedar Falls, IA (US)

(72) Inventor: Jane ALLEN, Cedar Falls, IA (US)

(21) Appl. No.: 15/615,542

(22) Filed: Jun. 6, 2017

(51) Int. Cl. A61M 25/00

(52) U.S. Cl. CPC A61M 25/0021; A61M 2005/1403; A61M 25/0068

(57) ABSTRACT

A catheter with a dedicated flushing port has a flushing port that allows the practitioner to irrigate an inserted catheter to remove blood clots and other substances from the patient without removing the drainage tube. A holder keeps the flushing port clean and while not in use. A drainage bag with a large spout is provided to allow easy empting of the drainage bag. In one embodiment, the flushing port is integrated as part of the catheter. In another embodiment, the flushing port is part of the drainage tubing and in another embodiment, the flushing port is provided as an attachment piece that fits between the catheter and the drainage tubing. The drainage bag has a handle that allows the user to tip the bag to empty.

### BACKGROUND OF THE INVENTION

The use of a catheter to drain the bladder has been known for over 3500 years. The word catheter is of Greek origin meaning to “let down” and referred to any device that was inserted into the body. There is documentation that ancient Syrians used reeds inserted into the urinary track to relieve a blocked bladder. Benjamin Franklin invented a silver catheter that he used to help his older brother that suffered from kidney stones.

Infection was a common but serious problem with these catheters. Eventually, the medical community came to realize that sterilization is extremely important whenever introducing anything into a human body. Additionally, flexible catheters were safer and more comfortable, but were more difficult to produce until advances in science gave us suitable materials. Modern urinary catheters are very flexible and come sterilized until ready for use.

In use, catheters may need to be irrigated to cleanse the bladder, tubing and to remove any blood clots that may form. In order to be irrigated, the user must disconnect an irrigation bag and then inject a sterile saline solution to flush the catheter system and then reconnect the irrigation bag. This procedure presents additional risks for infection and contamination when using the catheter. There is a need for a catheter that reduces the risk for infection by allowing the catheter to be irrigated and flushed without having to disconnect and reconnect the irrigation tube.

### SUMMARY OF THE INVENTION

A catheter with a dedicated flushing port has a flushing port that allows the practitioner to irrigate an inserted catheter to remove blood clots and other substances from the patient without removing the drainage tube. A holder keeps the flushing port clean and while not in use. A drainage bag with a large spout is provided to allow easy empting of the drainage bag. In one embodiment, the flushing port is integrated as part of the catheter. In another embodiment, the flushing port is part of the drainage tubing and in another embodiment, the flushing port is provided as an attachment piece that fits between the catheter and the drainage tubing. The drainage bag has a handle that allows the user to tip the bag to empty.

Other features and advantages of the instant invention will become apparent from the following description of the invention which refers to the accompanying drawings.

### BRIEF DESCRIPTION OF THE DRAWINGS

FIG. 1 is an illustration of prior art catheter setup using a syringe and flushing tray.

FIG. 2 is an illustration of a catheter with dedicated flushing port setup according to an embodiment of the invention.

FIG. 3 is an illustration of the catheter with dedicated flushing port shown in FIG. 1.

FIG. 4 is an illustration of the catheter with dedicated flushing port shown in FIG. 3 with flushing port in a storage configuration.

FIG. 5 is an illustration of a catheter with dedicated flushing port according to an embodiment of the invention.

FIG. 6 is an illustration of the catheter with dedicated flushing port shown in FIG. 5 with flushing port in a storage configuration.

FIG. 7 is an illustration of the catheter with dedicated flushing port shown in FIG. 2 with an irrigation syringe inserted in the flushing port

FIG. 8 is an illustration of the catheter with a with flushing port according to an embodiment of the invention.

FIG. 9 is a detailed view of the drainage bag shown in FIG. 2.

FIG. 10 is a view of the drainage bag shown in FIG. 9 with sealable drainage spout in an open configuration

FIG. 11 is a view of a two--way catheter with dedicated flushing port according to an embodiment of the invention.

FIG. 12 is a view of a one-way catheter with dedicated flushing port according to an embodiment of the invention.

FIG. 13 is front view of the flushing port shown with holder shown in FIG. 4.

FIG. 14 is a perspective view of the flushing port with holder shown in FIG. 4.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

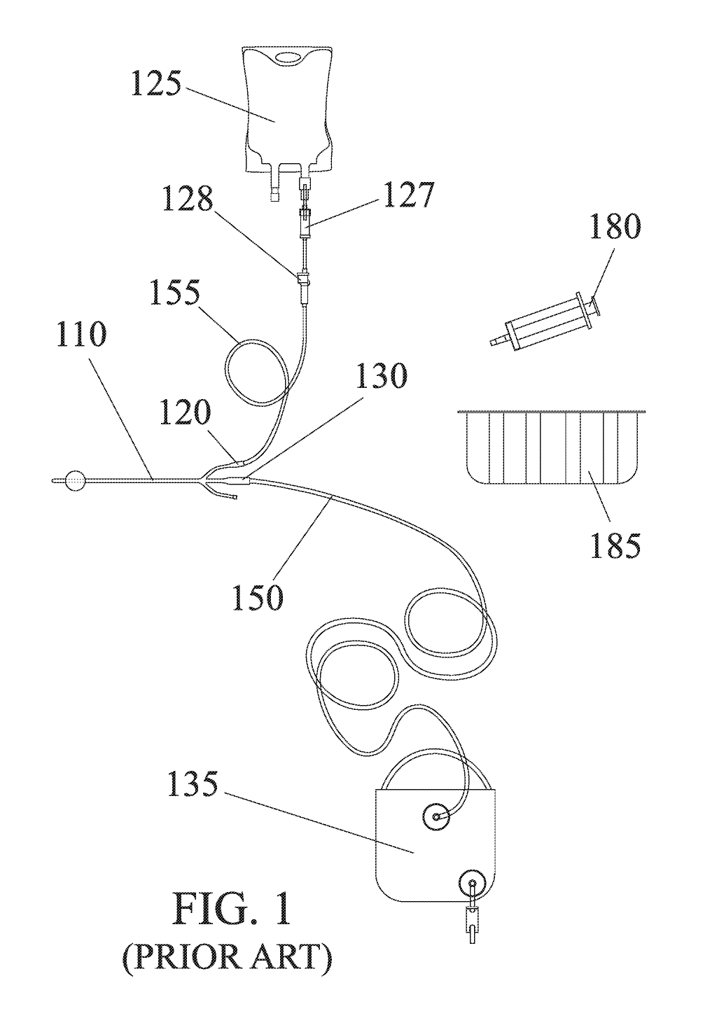

FIG. 1 shows a prior art catheter system in a typical setup with a continuous bladder irrigation bag (CBI) 125 attached using an irrigation tubing 155 which is attached to an irrigation port 120 disposed on a catheter 110. A drip chamber 127 and roller clamp 128 is typically used to control the flow of the irrigant. A drainage tubing 150 is attached to a drainage port 130 on catheter 110. Drainage tubing 150 is attached to catheter 110 and a drainage bag 135 is used to collect the urine when properly inserted into the urinary track of a patient (not shown). During use, the catheter must be flushed to remove blood clots and other substances that can build up. In order to flush the system, drainage tubing 150 must be removed and carefully placed to keep it clean and sterile. An irrigation syringe 180 is used to flush the system with a sterile solution and a tray 185 is placed under drainage port 130 to catch the flushing fluids as they flush from the system. Great care must be used to keep from introducing any bacteria or infectious agents into the system during this procedure. Once flushed, drainage tubing 150 is reconnected until flushing is needed and the procedure is repeated. In general, flushing a prior art catheter is one of the most frequent sources of contamination and infection in the patient.

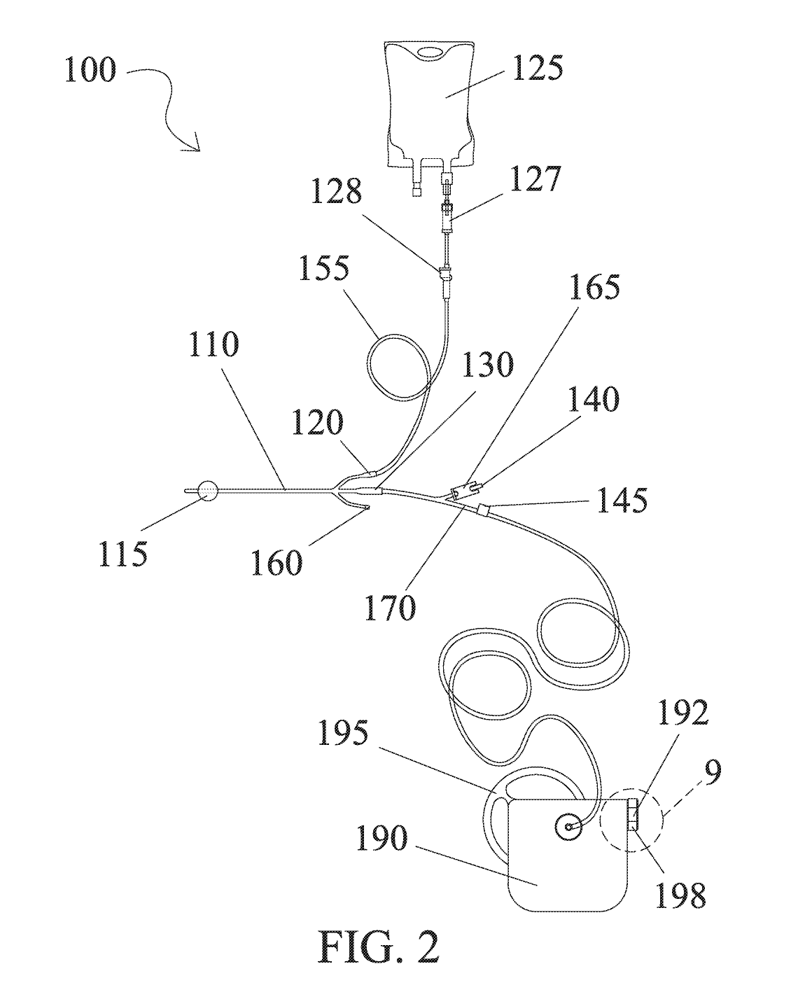

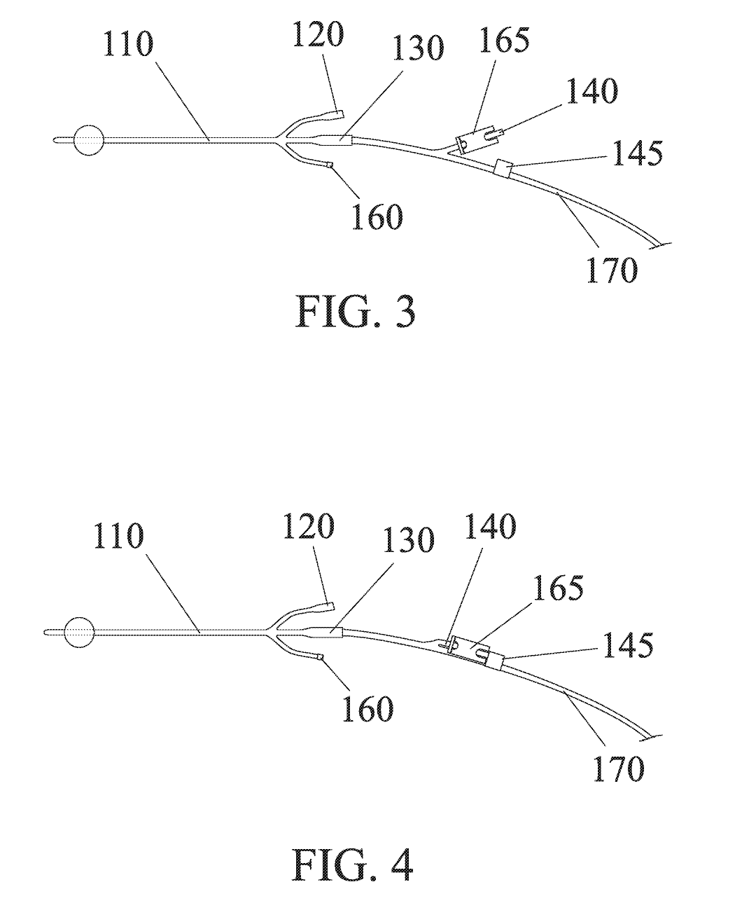

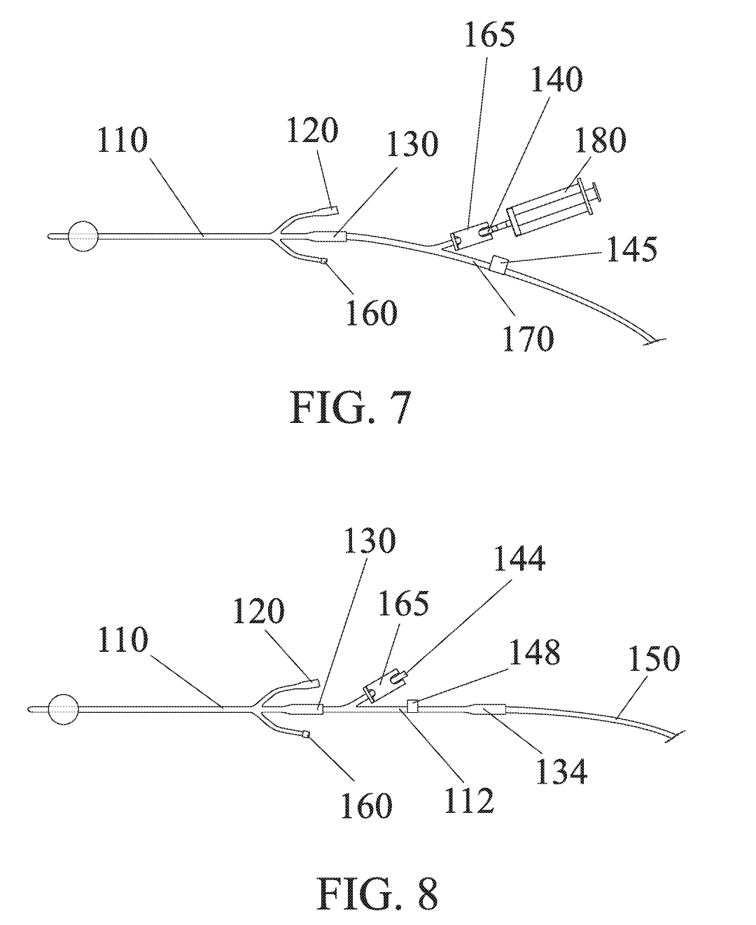

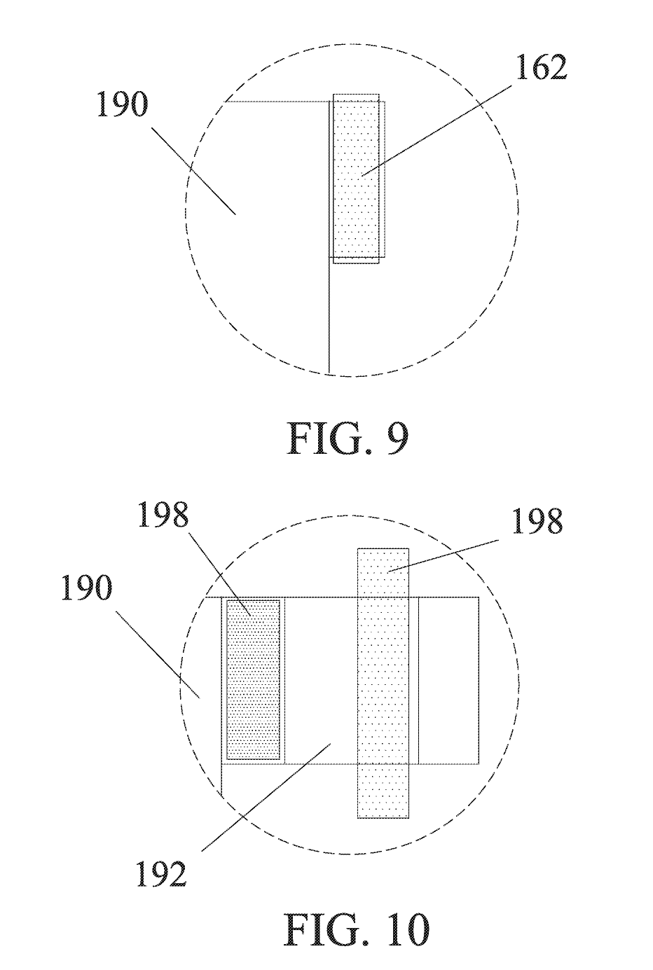

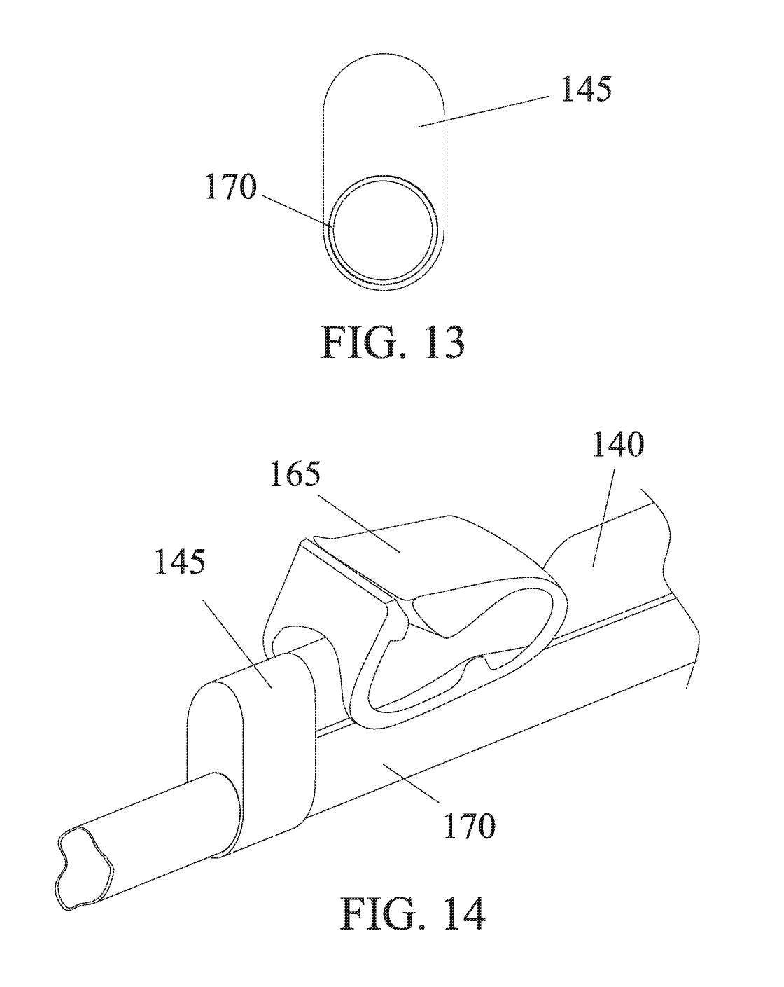

Referring to FIGS. 2-4, 7, 9-10 and 13-14, a catheter with dedicated flushing port system 100 is shown using 3-way catheter 110 has an end which is inserted in a patient (not shown) and held in place with an inflatable balloon portion 115 as is known in the art. Inflatable balloon portion 115 is inflated and deflated using a balloon fill port 160. In use, continuous bladder irrigation bag 125 is connected to irrigation port 120 to supply the irrigant using irrigation tubing 155 along with drip chamber 127 and roller clamp 128 as described above. A catheter drainage tube with dedicated flushing port 170 is connected to drainage port 130 on catheter 110. A dedicated flushing port 140 is provided to allow the user to flush catheter 110 without the necessity of disconnecting and then reconnecting catheter drainage tube with dedicated flushing port 170. A drainage bag 190 is connected using catheter drainage tubing with dedicated flushing port 170.

Drainage bag 190 has a sealable drainage spout 192 that is sealed using a hook and loop fastener 198. Sealable drainage port 192 is large enough to easily allow blood clots and other collected substances to be removed by opening sealable drainage spout 192 and tipping drainage bag 190 using a handle 195. Once drained, sealable drainage spout 192 is resealed. Of course other resealable means may be used to seal drainage bag 190 as long as sealable drainage spout 192 is resealable. A flushing port holder 145 allows dedicated flushing port 140 to be sanitarily stored while not in use. A clamp 165 is used to clamp off dedicated flushing port 140 when not in use.

When flushing is required, dedicated flushing port 140 is removed from holder 145 and irrigation syringe 180 is filled with irrigant solution such as a saline solution and then inserted in dedicated flushing port 140 where the irrigant is injected and then clamp 165 is used to clamp off dedicated flushing port 140. Of course other clamping means may be used to control the flow fluids as is known in the art. The flushing fluid irrigates the urinary track of the patient and then flows out through drainage port 130 and is directed down through catheter drainage tube with dedicated flushing port 170 and into drainage bag 135 without the need to open the system up, thus reducing the likelihood of external contamination and infection. The system may be flushed as needed by repeating the procedure. When flushing is complete, dedicated flushing port 140 is placed in holder 145 until needed.

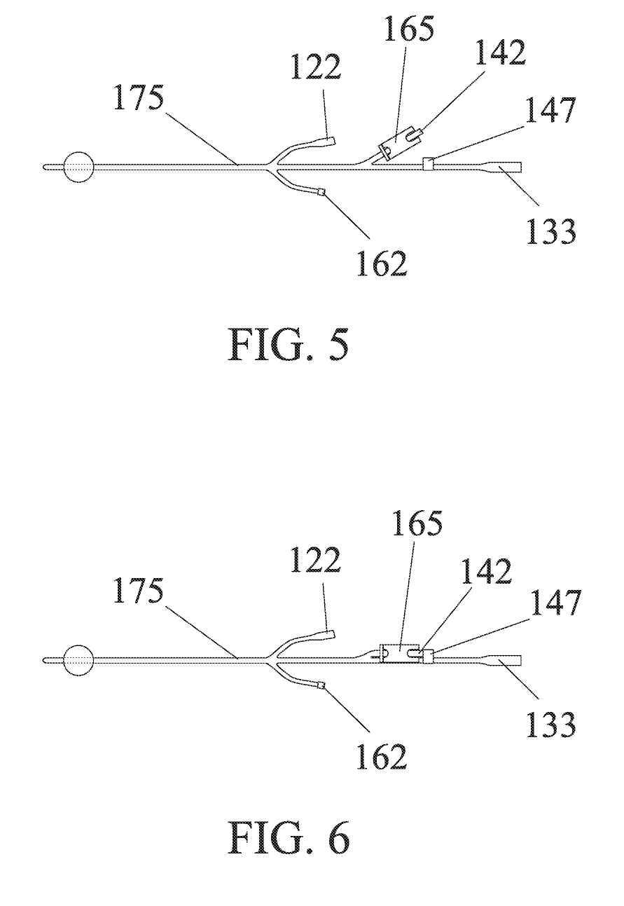

Referring now to FIGS. 5 and 6, a catheter with integral dedicated flushing port 175 is shown having an elongated drainage port portion 133 with a dedicated flushing port 142 and a holder 147 to secure dedicated flushing port 142 when not in use. Again clamp 165 is used to control fluid flow. An irrigation port 122 and balloon fill port 162 are provided to be used as described above. FIG. 6 illustrates the non-use storage position with dedicated flushing port 142 retained in holder 147.

Now referring to FIG. 8, a flushing port attachment 112 is attached to catheter 110 using drainage port 130 and then drainage tubing 150 is attached to a drainage port 134 located on flushing port attachment 112. A dedicated flushing port 144 is provided to allow the user to flush the system without detaching drainage tubing 150 as described above. As discussed above, flushing port 144 is removed from a holder 148 which allows the user to flush the system without having to detach the drainage tube.

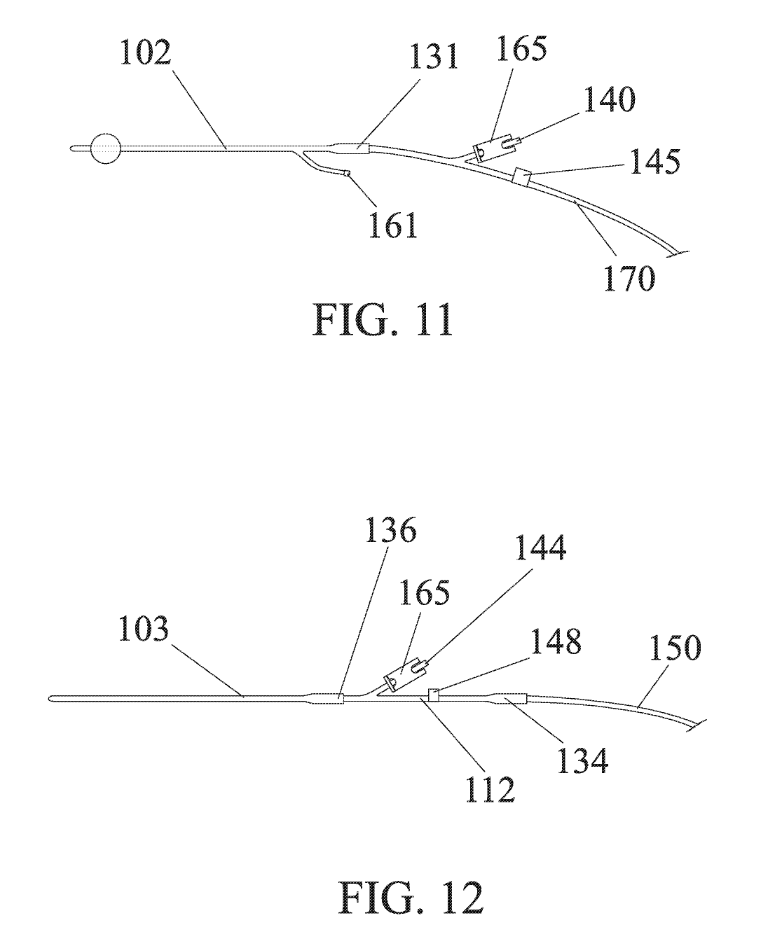

Referring to FIG. 11, a two-way catheter 102 is shown having a drainage port 131 and a balloon fill port 161. Catheter drainage tube with dedicated flushing port 170 is inserted in drainage port 131. As discussed above, flushing port 140 is removed from holder 145 and allows the user to flush the system without having to detach the drainage tube.

Now referring to FIG. 12, a one-way catheter 103 is shown having a drainage port 136. Flushing port attachment 112 is attached to catheter 103 using drainage port 134 and then drainage tubing 150 is attached to a drainage port 134 located on flushing port attachment 112. Dedicated flushing port 144 is provided to allow the user to flush the system without detaching drainage tubing 150 as described above.

Signs and Symptoms of a Blocked Catheter
No urine flow from the catheter.
Suprapubic distention and lowerabdominal pain becoming more pronounced as the bladder fills.
Urine leaking around the catheter (bypassing).
Vaso-vagal symptoms may develop i.e, sweating, tachycardia and hypotension if the blockage is unrelieved.
Autonomic dysreflexia in Spinal Cord Injured (SCI) patients.

Optimal Outcome
The dots are removed from bladder and the urine is draining freely.

Sub-Optimal Outcomes
Over distention of the bladder.
Inability to unblock the IUC and requiring catheter replacement.
Development of a catheter associated urinary tract infection (CAUTI) secondary to contamination during the procedure and break in the closed urinary drainage system.

Equipment
Alcohol based hand rub.
70% alcohol swabs.
1 bottle 500 mL sterile sodium chloride 0.9% (Normal Saline).
Disposable under pad.
Non-sterile receptacle.
Irrigation set (60 ml syringe and wide top bottle to hold irrigant).
1 pair of sterile gloves
Personal Protective Equipment (PPE) protective eyewear, plastic apron/disposable gown and gloves (if required by policy)

Procedure
Perform hand hygiene.
Explain procedure to patient, obtain consent and identify allergies.
Ensure patient privacy.
Position patient in supine position, allow for easy access.
Place a disposable sheet under the work area.
Place non-sterile jug/receptacle nearby.
Clamp off CBI on irrigation tubing.
Perform hand hygiene.
Pour sterile chloride 0.9% into irrigation receptacle.
Perform hand hygiene.
Put on gloves.
Draw up 50 mL of sodium chloride 0.9% in syringe.
Remove flushing port from holder.
Clean flushing port well with the 70% alcohol swabs and discard swab.
Insert syringe into flushing port, and unclamp clamp.
Using 50 mL volumes of sodium chloride 0.9%, irrigate catheter by flushing in and drawing back on the plunger to evacuate any clot or debris.

Warn the patient that this will be painful/uncomfortable. If resistance is encountered reasonable pressure can be used (except following renal transplant or bladder surgery).
Empty each syringe directly into the non-sterile jug/receptacle or allow to flow down drainage tubing.
Continue to irrigate with 50 mL volumes until drainage runs clear or a clot free return is achieved.
Clamp off flushing port using clamp.
Clean flushing port with alcohol.
Replace flushing port into holder.
Recommence continuous bladder irrigation by unclamping tubing.
Remove disposable sheets and ensure that the patient is comfortable.
Remove personal protection equipment (PPE).
Dispose waste according to local policy.
Perform hand hygiene.
Calculate the difference between volume in and volume returned.

Document outcome in patient clinical progress notes including:
Date and time of procedure.
Indication for the procedure including the patients clinical signs and symptoms.
Outcome i.e. color and type of drainage, presence of clots and patients tolerance of the procedure.
Record on the fluid balance chart volume in the volume return.

Although the instant invention has been described in relation to particular embodiments thereof, many other variations and modifications and other uses wall become apparent to those skilled in the art.

What is claimed is:

1. A catheter with an integral flushing port comprising;
a catheter;
said catheter having an insertion end;
said catheter having a drainage port;
a flushing port;
said flushing port being disposed between said insertion end and said drainage port; and
said flushing port adapted to selectively introduce flushing irrigant into said catheter.

2. The catheter with an integral flushing port according to claim 1 further comprising:
a drainage tube;
said drainage tube having an insert end and a drainage end;
a drainage bag;
said insert end being inserted in said drainage port; and
said drainage end being attached to said drainage bag.

3. The catheter with an integral flushing port according to claim 2 further comprising:
a holder proximally disposed to said flushing port; and
said holder adapted to removably hold said flushing port in place when not in use.

4. The catheter with an integral flushing port according to claim 2 wherein said drainage bag has a sealable drainage spout; said sealable drainage spout having an opening size adapted to drain both liquid and solid contents.

5. The catheter with an integral flushing port according to claim 4 wherein said sealable drainage spout has a hook and loop closure.

6. The catheter with an integral flushing port according to claim 1 wherein said catheter has an elongated drainage port portion; said flushing port being disposed on said elongated drainage portion.

7. The catheter with an integral flushing port according to claim 1 wherein said catheter is a three-way catheter.

8. The catheter with an integral flushing port according to claim 1 wherein said catheter is a two--way catheter.

9. The catheter with an integral flushing port according to claim 1 wherein said catheter is a one-way catheter.

10. The catheter with an integral flushing port according to claim 5 further comprising a handle disposed on said drainage bag wherein said drainage bag is emptied by tipping using said handle .

11. A catheter with a flushing port comprising;
a catheter;
said catheter having an insertion end and a drainage port;
a catheter drainage tube;
said catheter drainage tube having a catheter insertion end and a drainage bag end;
said catheter insertion end being removably disposed in said drainage port;
a flushing port proximally disposed on said catheter insertion end of said catheter drainage tube; and
said flushing port adapted to selectively introduce flushing irrigant into said catheter.

12. The catheter with a flushing port according to claim 11 further comprising:
a holder proximally disposed to said flushing port; and
said holder adapted to removably hold said flushing port in place when not in use.

13. The catheter with a flushing port according to claim 12 further comprising:
a drainage bag; and
said drainage bag end being removably attached to said drainage bag.

14. The catheter with a flushing port according to claim 13 wherein said drainage bag has a sealable drainage spout; said sealable drainage spout having an opening size adapted to drain both liquid and solid contents.

15. The catheter with a flushing port according to claim 14 wherein said sealable drainage spout has a hook and loop closure.

16. A catheter with a flushing port comprising;
a catheter;
said catheter having a catheter drainage port;
a drainage port attachment;
said drainage port attachment being removably disposed within said catheter drainage port;
said drainage port attachment having an attachment drainage port;
a flushing port disposed on said drainage port attachment; and
said flushing port adapted to selectively introduce flushing irrigant into said catheter.

17. The catheter with a flushing port according to claim 16 further comprising:
a holder proximally disposed to said flushing port; and
said holder adapted to removably hold said flushing port in place when not in use.

18. The catheter with a flushing port according to claim 17 further comprising:
a drainage tube;
said drainage tube having an insert end and a drainage end;
a drainage bag;
said insert end being inserted in said attachment drainage port; and
said drainage end being attached to said drainage bag.

19. The catheter with an integral flushing port according to claim 18 wherein said drainage bag has a sealable drainage spout; said sealable drainage spout having an opening size adapted to drain both liquid and solid contents.

20. The catheter with an integral flushing port according to claim 19 wherein said sealable drainage spout has a hook and loop closure.
